(19) United States
(12) Patent Application Publication
(10) Pub. No.: US 2008/0051901 A1
(43) Pub. Date: Feb. 28, 2008

(54) Spinal Prosthesis with Multiple Pillar Anchors

(75) Inventors: Malan de Villiers, Irene (ZA); David Hovda, Mountain View, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: SpinalMotion, Inc., Mountain View, CA (US)

(21) Appl. No.: 11/829,056

(22) Filed: Jul. 26, 2007

Related U.S. Application Data
(60) Provisional application No. 60/820,770, filed on Jul. 28, 2006.

(51) Int. Cl. A61F 2/44; A61B 17/56

(52) U.S. Cl. 623/17.16; 606/72

(57) ABSTRACT

An intervertebral prosthesis includes a first component adapted to engage a first vertebra and a second component adapted to engage a second vertebra. A row of pillars is disposed on at least one of the components. The row of pillars is adapted to enter a groove and anchor the prosthesis in the intervertebral space. The pillars can be shaped and spaced apart to provide gaps so that bone can grow into the gaps. Additional rows of pillars can be disposed on the components and adapted to enter additional grooves formed in the vertebra.

### CROSS REFERENCE TO RELATED APPLICATIONS

The present non-provisional application claims the benefit under 35 USC 119e of U.S. Appl. No. 60/820,770 (attorney docket no. 022031-003100US), entitled “Spinal Prosthesis with Multiple Pillar Anchors”, filed on July 28, 2006, the full disclosure of which is incorporated herein by reference.

### BACKGROUND OF THE INVENTION

1. Field of the Invention

This invention relates to medical devices and methods. More specifically, the invention relates to restoration of spinal motion with a prosthetic disc for intervertebral insertion, such as in the lumbar and cervical spine.

In the event of damage to a lumbar or cervical intervertebral disc, one possible surgical treatment is to replace the damaged disc with an intervertebral disc prosthesis. Several types of intervertebral disc prostheses are currently available. One type available under the trademark. SB Charite (DePuy Spine, a division of Johnson & Johnson, New Brunswick, N.J.), includes upper and lower prosthesis plates or shells which engage the adjacent vertebral bodies with a low friction core between the plates. [See EP 1142544A1 and EP 1250898A1] Many prosthetic discs use protruding anchors to anchor the endplates to the adjacent vertebra, for example, an elongate anchor adapted to enter a groove cut into a vertebra as described in U.S. Pat. No. 4,863,477. While elongate fins, keels and other anchors have generally been successful in anchoring endplates to vertebra, clinical trials with large numbers of patients have shown that in rare cases the endplates of the implanted prosthetic disc can slip, causing patient discomfort and requiring surgical intervention. Another rare complication can arise with the elongate anchors when prosthetic discs are placed in adjacent intervertebral spaces on opposite ends of a vertebra, referred to as “stacking”. The vertebra positioned between the adjacent prosthetic disks can split, possibly as a result of grooves cut in the same plane on the upper and lower surfaces of the vertebra. This complication is also undesirable and typically requires surgical intervention.

Another prosthetic approach has been to fuse the vertebrae, for example with transforaminal lumbar interbody fusion (TLIF) surgery or posterior lumbar interbody fusion (PLIF) surgery. Fusion surgery generally requires at least partial removal of one or more facet joints, bone grafting, and support with a fusion cage to stop the motion at that segment. Although the fusion cages can be inserted from the back of the patient, such prostheses generally do not provide a flexible joint at the damaged disc site or other implant site. Thus a potential disadvantage of these fusion approaches is that spinal motion is not restored at the intervertebral joint.

In light of the above, it would be desirable to provide improved prostheses, particularly surgical prostheses which at least partially restore motion and provide consistent attachment of the prosthetic endplates to the vertebrae.

2. Description of the Background Art

U.S. patent application under U.S. Patent Publication Nos. 2002/0035400A1 and 2002/0128715A1 describe disc implants which comprise opposing plates with a core between them over which the plates can slide. Expandable intervertebral prostheses are described in U.S. Appl. No. 60/744710 (attorney docket no. 022031-001900US), entitled “Spinal Disc Arthroscopy,” filed on Apr. 12, 2006; and U.S. Appl. No. 60/746731 (attorney docket no. 022031-001910US), entitled “Spinal Disk Arthroscopy,” filed on May 8, 2006, the full disclosures of which are incorporated herein by reference. Other patents related to intervertebral disc prostheses include U.S. Pat. Nos.: 4,759,766; 4,863,477; 4,997,432; 5,035,716; 5,071,437; 5,258,031; 5,370,697; 5,401,269; 5,507,816; 5,534,030; 5,556,431; 5,674,296; 5,676,701; 5,676,702; 5,702,450; 5,797,909; 5,824,094; 5,865,846; 5,989,291; 6,001,130; 6,022,376; 6,039,763; 6,096,038; 6,139,579; 6,156,067; 6,162,252; 6,315,797; 6,348,071; 6,368,350; 6,416,551; 6,592,624; 6,607,558; 6,706,068; 6,740,118; and 6,936,071. Other patent applications related to intervertebral disc prostheses include U.S. Patent Publication Nos.: 2001/0016773; 2002/0035400; 2002/0128715; 2003/0009224; 2003/0074076; 2003/0100951; 2003/0135277; 2003/0191536; 2003/0208271; 2003/0199982; 2004/0030391; 2004/0073312; 2004/0143270; 2004/0176843; 2005/0043800; 2005/0085917; 2005/0107881; 2005/0149189; 2005/0192586; 2005/0261772; and 2006/0041313. Other related patents and applications include: WO 01/01893A1, WO 2005/053580, EP 1344507, EP 1344506, EP 1250898, EP 1306064, EP 1344508, EP 1344493, EP 1417940, EP 1142544, and EP 0333990.

### BRIEF SUMMARY OF THE INVENTION

The present invention provides an implanted intervertebral prosthesis which restores motion, provides improved attachment of the prosthesis to the adjacent vertebrae, and may decrease the possibility of vertebral splitting where multiple adjacent implants are used. The prosthesis includes first and second components adapted to attach to a first vertebra and a second vertebra, respectively, that define the intervertebral space. The prosthesis includes at least one row of pillars, posts, or other elongate anchor, disposed along a row on at least one of the components. The row of pillars is adapted to enter into a groove formed in one of the adjacent vertebra, so that once the row of pillars is placed in the groove, the row of pillars anchors the component to the adjacent vertebra. The first and second components are adapted to articulate so that motion is restored between the first and second vertebrae while the row of pillars anchors the prosthetic disc in the intervertebral space.

A row of pillars disposed over a length can provide better attachment to bone than a single elongate anchor of comparable length. For example, the row of pillars can provide a greater surface area to attach to the bone over the length. Also, the pillars can be spaced apart to provide gaps so that bone can grow between the pillars to rigidly anchor the pillars to the vertebra. In addition, a row of pillars may provide some flexure or bending of the pillars so that mechanical stress on vertebra near the groove is decreased as compared to a solid anchor of the same length, thereby decreasing the possibility of vertebral splitting. For example, intervertebral prostheses are often driven into the intervertebral space with force by striking the implant, and the row of pillars can dampen and decrease potentially traumatic force transmitted to the vertebrae as the implant is driven into the intervertebral space.

The pillars can be shaped and arranged in many ways on the prosthetic disc to anchor the disc in the intervertebral space. The pillars typically have a base attached to one of the components and the pillar extends from the base to the tip, often with substantially the same cross sectional shape. The pillars can be disposed in a first row on the first component and a second row on the second component so that each component is attached to an adjacent vertebra with pillars to hold the prosthetic disc in the intervertebral space. Additional rows of pillars can also be used, for example two rows on each component. The pillars are often separated by a distance to provide a gap between the pillars so that bone can grow into the gap between the pillars and rigidly hold the component. The number of pillars disposed in a row can be selected so that the gap is present between each of the pillars. The rows of pillars can be offset from each other to avoid cutting a vertebra on along the same plane on each opposing end of the vertebra in cases where two prosthesis are used in adjacent intervertebral spaces, i.e. stacked.

In one aspect, embodiments of the present invention comprise an intervertebral prosthesis. The intervertebral prosthesis comprises a first component adapted to engage a first vertebra, and a second component adapted to engage a second vertebra. A row of pillars is disposed on at least one of the upper component or the lower component. The row of pillars is adapted to enter a groove formed in the first vertebra or the second vertebra. The upper component and the lower component are adapted to form an articulated joint between the vertebrae. This articulated joint can be formed by direct engagement between the upper component and the lower component, or by each component engaging an intermediate member, for example a sliding core, disposed between the two components to form the articulate joint.

In many embodiments, the pillars are shaped and arranged to anchor the component to the vertebra. For example, a row of pillars can include a gaps between the pillars to permit bone growth between the pillars. The size and number of the pillars can be selected to provide the gap between the pillars, for example the row of pillars can comprise from about 5 to 20 pillars. Typically, each pillar of the row will have a base attached to the first component or the second component so that the pillar is firmly attached to the component, and each pillar extends from the base to a tip. Each pillar has a height, for example from the base to the tip, and several pillars have substantially the same height so that the row of pillars can fit tightly in the groove and engage the groove. Each pillar has a maximum cross sectional width at the base of the pillar, and the height of each pillar is typically at least the maximum cross section width at the base, often at least twice the maximum cross sectional width at the base, and ideally from about 3 to 8 times the maximum cross sectional width at the base. Also, several of the pillars will have a similar cross-sectional geometry so that the pillars can fit tightly into the groove.

In specific embodiments, the pillars are shaped and arranged to facilitate insertion into the groove so that the pillars of a row will fit tightly in the groove, for example where the row of pillars is driven into the groove with force. For example, the row of pillars can include pillars of increasing height along the row so that the row presents pillars of increasing height to the groove as the row is advanced distally into the groove. At least some of the pillars have a tapered cross sectional width which increases in a proximal direction so that the pillar initially presents a narrow cross sectional width to the groove and subsequently presents a wider cross sectional width to the groove as the pillar is advanced distally into the groove. At least some of the pillars have a vertical recess on a distal surface. The vertical recess provides space into which bone can grow to anchor the implant, and the recess does not initially engage bone recess as the pillar is advanced distally into the groove.

In the illustrated embodiments several of the pillars extend vertically, although several of the pillars can be inclined. For example the pillars can be inclined proximally to facilitate insertion into the groove. Also, the pillars can be inclined away from a vertical plane extending in proximal and distal directions to decrease cut depth into the vertebra.

In some embodiments, an intervertebral prosthesis comprises an upper component that has a row of upper pillars disposed thereon, and a lower component that has a row of lower pillars disposed thereon. The upper row of pillars is arranged to enter a groove in an upper vertebra. The lower row of pillars is arranged to enter a groove in a lower vertebra. The upper component and the lower component are adapted to engaged each other or an intermediate member to form an articulate joint.

In specific embodiments, the pillars of the upper and lower rows are shaped and arranged to anchor the components, and additional rows of pillars can be used. For example, both the upper and lower rows of pillars can include gaps to permit bone growth between the pillars. As described above, from about 5 to 20 pillars can be disposed in each row to provide gaps between the pillars. At least one component can include two rows of pillars adapted to enter two parallel grooves in one of the vertebrae. For example, an upper component can include a single row of pillars, and the lower component can include two rows of pillars so that the upper row is adapted to enter a single groove cut in a vertebra and each of the two lower rows is adapted to enter a groove in the lower vertebra. The rows of pillars can offset from each other to avoid vertebral splitting. For example, the prosthetic disc can include a midline and each row can be disposed on an opposite side of the midline to offset the upper row from the lower row. Also, one of the components can have two rows of pillars disposed on opposite sides of the midline, and the other component can have a single row of pillars disposed on the midline so that the rows are offset.

In another aspect, embodiments of the present invention comprise method for anchoring an intervertebral prosthesis within an intervertebral space between a pair of vertebral bodies. The method comprises cutting a groove in at least one of the vertebral bodies, and introducing the prosthesis into the intervertebral space. A plurality of pillars on the prosthesis enters the groove to anchor the prosthesis to at least one of the vertebrae.

In some embodiments, the components are arranged to articulate while the pillars are introduced into the groove, for example after the prosthesis is partially inserted into the intervertebral space and released to minimize distraction. A groove can be cut in each of the vertebral bodies so that each vertebral body is adapted to receive pillars.

### BRIEF DESCRIPTION OF THE DRAWINGS

FIG. 1 shows an intervertebral prosthesis implanted between adjacent vertebrae according to an embodiment of the present invention;

FIG. 2A shows an isometric view of the intervertebral prosthesis of FIG. 1 with several pillars adapted for insertion into a vertebra according to an embodiment of the present invention;

FIG. 2B shows a side view of the intervertebral prosthesis as shown in FIGS. 1 and 2A according to an embodiment of the present invention;

FIG. 2C shows a front view of the intervertebral prosthesis as shown in FIGS. 1, 2A and 2B according to an embodiment of the present invention;

FIG. 3A shows an end view of an intervertebral prosthesis with rows of pillars offset from a midline of the prostheses according to an embodiment of the present invention;

FIG. 3B shows an end view of an intervertebral prostheses with two rows of pillars on the top end plate and a single row of pillars on the bottom endplate according to an embodiment of the present invention;

FIG. 4A schematically illustrates pillars inclined away from a midline on an intervertebral implant according to an embodiment of the present invention.;

FIG. 4B schematically illustrates pillars inclined proximally on an intervertebral implant according to an embodiment of the present invention.

### DETAILED DESCRIPTION OF THE INVENTION

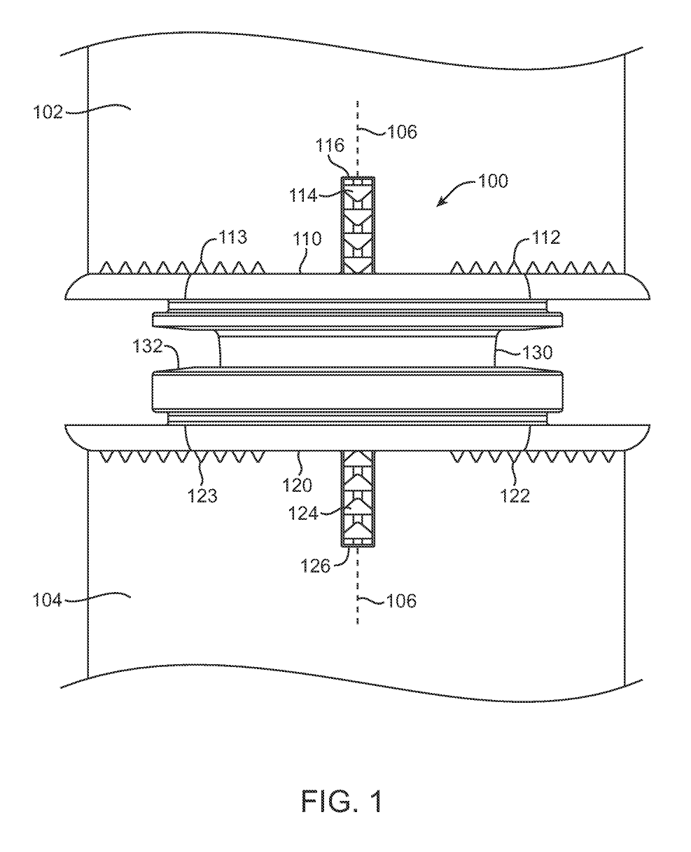

FIG. 1 shows an intervertebral prosthesis 100 implanted between adjacent vertebrae according to an embodiment of the present invention. An upper vertebra 102 is located adjacent a lower vertebra 104. Intervertebral prosthesis 100 is located between upper vertebra 102 and lower vertebra 104. Prosthesis 100 includes an upper endplate 110 adapted to bear against and connect to upper vertebra 102. Prosthesis 100 is symmetrically disposed about a midline 106, and midline 106 is generally aligned with a midline passing through the upper and lower vertebrae. Serrations 113 and 112 are provided on upper endplate 110 to attach the upper endplate to the upper vertebra. Serrations 113 and 112 are generally symmetrically disposed on opposite sides of midline 106. A lower endplate 120 is adapted to bear against and connect to lower vertebra 104. Serrations 122 and 123 are provided on lower endplate 120 to attach the lower endplate to the lower vertebra. Serrations 122 and 123 are generally symmetrically disposed about opposite sides of midline 106. A row of pillars 114 is provided on upper endplate 110 to attach the endplate to the upper vertebra. A groove 116 is formed in upper vertebra 102 to receive row of pillars 114. As seen in FIG. 1, row of pillars 114 has been inserted into groove 116. A row of pillars 124 is provided on lower endplate 120. A groove 126 is formed in lower vertebra 104 to receive row of pillars 124. As seen in FIG. 1, row of pillars 124 has been inserted into groove 126.

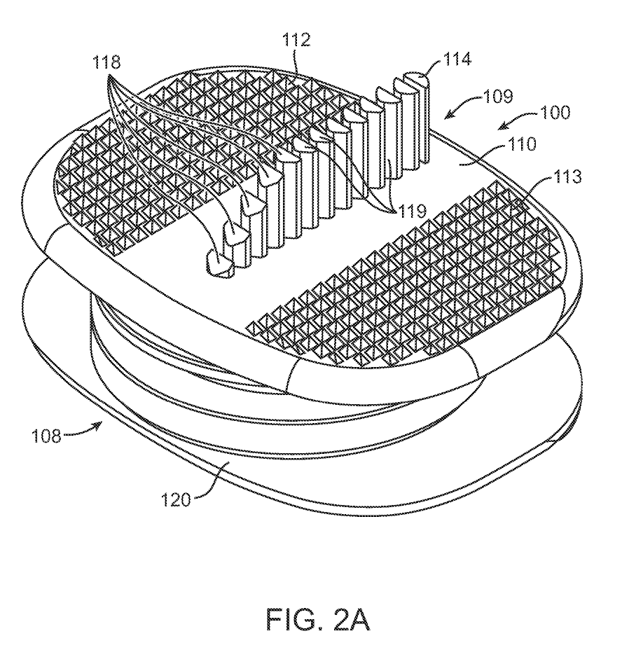

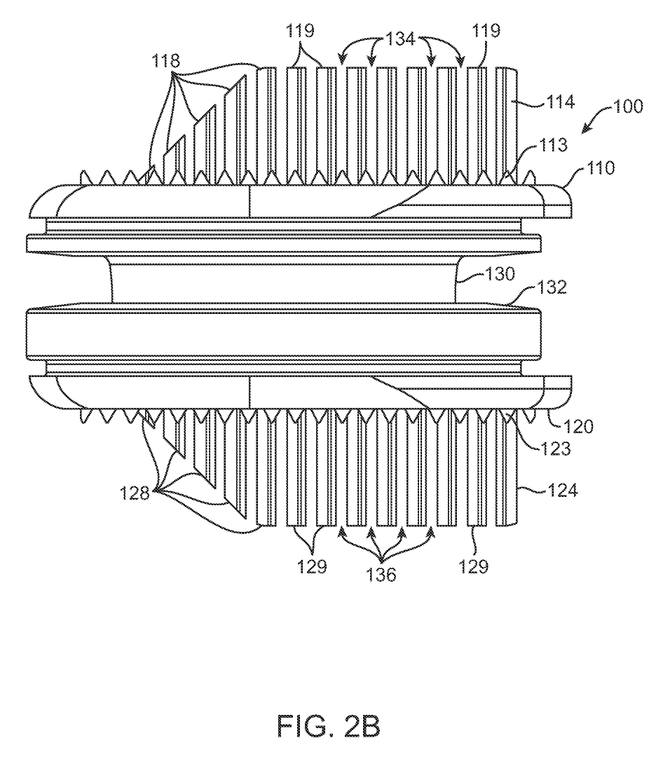

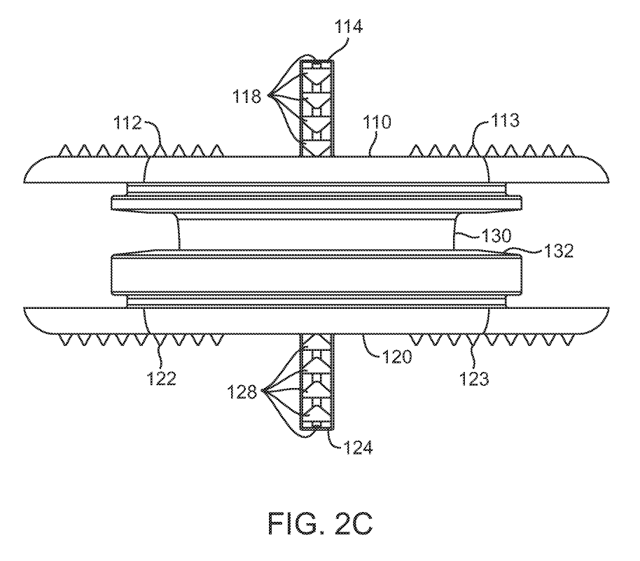

FIG. 2A shows an isometric view of intervertebral prosthesis 100 as shown in FIG. 1 with several pillars adapted for insertion into a vertebra according to an embodiment of the present invention. Prosthesis 100 includes a distal end 108 that leads as the implant is advanced into the intervertebral space and a proximal end 109 that follows distal end 108 as the implant is advanced into the intervertebral space. Row of pillars 114 includes several lead pillars 118. Lead pillars 118 are disposed near distal end 108 of upper plate 100 so that lead pillars 118 are inserted into groove 116 first as the prosthesis is inserted into the intervertebral space. Lead pillars 118 have an increasing height along the row so that lead pillars 118 can be easily inserted into the groove initially to help align the prosthesis with the groove. Each pillar has a base attached to endplate 110 and extends upward from the base toward a tip. Middle pillars 119 are disposed near the middle of the row of pillars and have the same height. Most of the pillars have the same cross sectional shape, for example triangular as shown in FIG. 2a, and the pillar extends vertically from the base with substantially the same cross sectional shape. However, the cross sectional shape of the pillar can assume many forms including square as with a pyramid shape pillar, circular as with a cylindrical pillar, square as with a rectangular pillar, elliptical, polygonal. Serrations 112 and 113 are disposed in rows that are parallel to the row of pillars 114. Each serration has a square base extending upward toward a tip, so that each serration has a pyramidal shape.

Upper endplate 110 and lower endplate 120 are adapted to articulate. As seen in FIG. 2A, a mobile core 130 is disposed between the two endplates. Core 130 is retained by retention ring 132 as described in U.S. application Ser. Nos. 10/855,817 and 10/855,253, both filed on May 26, 2004, the full disclosures of which are incorporated herein by reference. In alternate embodiments, the upper and lower plates directly engage each other with a ball and socket joint as described in U.S. Pat. Nos. 5,258,031 and 5,676,701, the full disclosures of which are incorporated herein by reference. In other embodiments, a core is locked in place between the endplates to provide a pivot surface as described in U.S. Pat. Nos. 5,314,477 and 6,936,071, the full disclosures of which are incorporated herein by reference.

FIG. 2B shows a side view of intervertebral prosthesis 100 as shown in FIGS. 1 and 2A according to an embodiment of the present invention. Pillars 114 are spaced apart with gaps 134. Gaps 134 permit bone growth between pillars 114 so that pillars 114 and plate 110 are held rigidly in place. Pillars 124 include lead pillars 128 that have an increasing height along row 124. Row 124 includes several middle pillars 129 having the same height. Row 124 includes gaps 136 between the pillars so that bone can grow between pillars 124 and rigidly hold the pillars and lower endplate 120 in place.

FIG. 2C shows a front view of the intervertebral prosthesis of FIGS. 1, 2A and 2B according to an embodiment of the present invention. Row 114 is disposed on upper endplate 110, and lead pillars 118 are connected to upper endplate 110. Row 124 is disposed on lower endplate 120, and lead pillars 128 are connected to lower endplate 120.

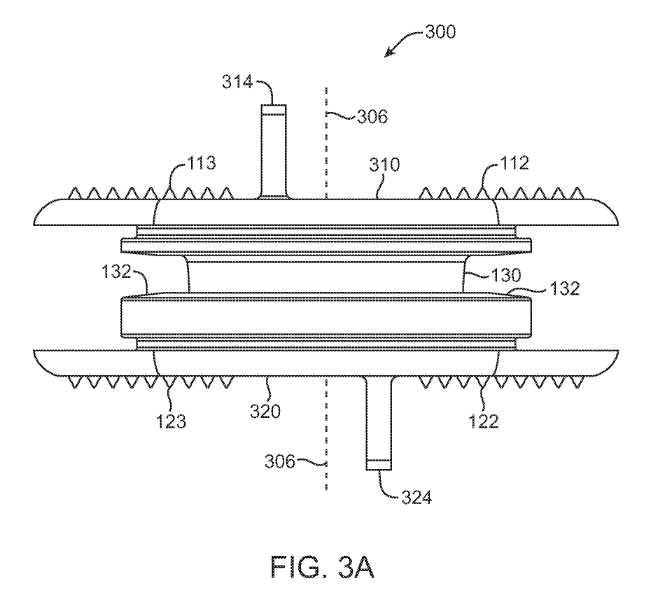

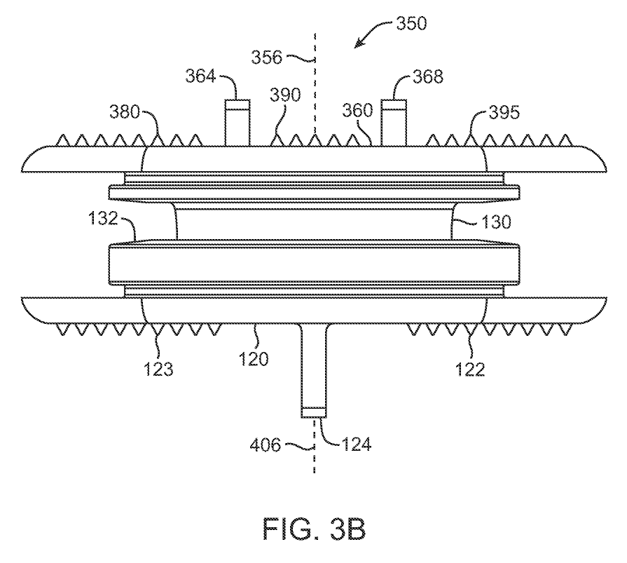

FIG. 3A shows an end view of a prosthesis 300 with rows of pillars offset from a midline of the prostheses according to an embodiment of the present invention, as described in U.S. Appl. No. 60/820,769 (attorney docket no. 022031-002000US), entitled “Spinal Prosthesis with Offset Anchors,” filed on Jul. 28, 2006, the full disclosure of which is incorporated herein by reference. Such an offset arrangement can be beneficial where adjacent prostheses are disposed between adjacent vertebra because such an arrangement preserves vertebral thickness so as to maximize vertebral strength and decrease the possibility of vertebral splitting between endplates on opposite sides of a vertebra. A midline 306 is disposed centrally on the prosthesis. An upper endplate 310 includes an upper row of pillars 314. Upper row of pillars 314 is offset from midline 306. Upper row of pillars 314 includes several lead pillars, several middle pillars and gaps as described above. A lower endplate 320 includes a lower row of pillars 324. Lower row of pillars 324 is offset from midline 306. Lower role of pillars 324 includes several lead pillars, several middle pillars and gaps as described above. Core 130 and a retention ring 132 are disposed between the endplates to provide articulation of the endplates, although articulated arrangements are possible as described above.

FIG. 3B shows an end view of an intervertebral prostheses 350 with two rows of pillars on the upper endplate and a single row of pillars on the lower endplate. This arrangement of the rows of pillars on the upper and lower endplates can preserve vertebral thickness and prevent vertebral splitting as described above. An upper endplate 360 includes a first row of pillars 364 and a second row of pillars 368. Each pillar of each row of pillars is attached to the endplate, includes several lead pillars and several middle pillars as described above. Row of pillars 364 and row of pillars 368 are offset from a midline 356 and symmetrically disposed about midline 356. Serrations 380, 390 and 395 are provided to attach endplate 360 to the upper vertebra. Rows of serrations 380 are disposed parallel to and peripheral to rows of pillars 364 and 368. Rows of serrations 390 are disposed parallel to and in between rows of pillars 364 and 368. Rows of serrations 395 are disposed peripheral to and parallel to rows of pillars 364 and 368. Lower endplate 120 with serrations 122, 123 and row of pillars 124 is described above with reference to FIGS. 1 to 2C.

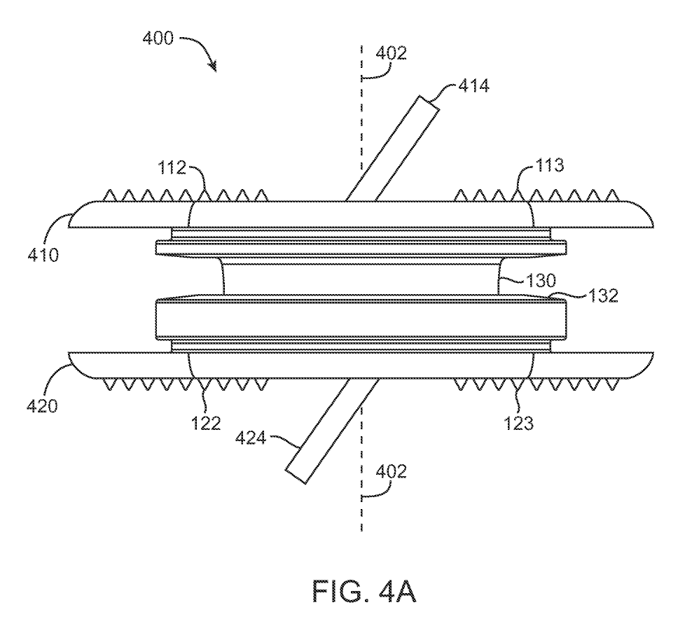

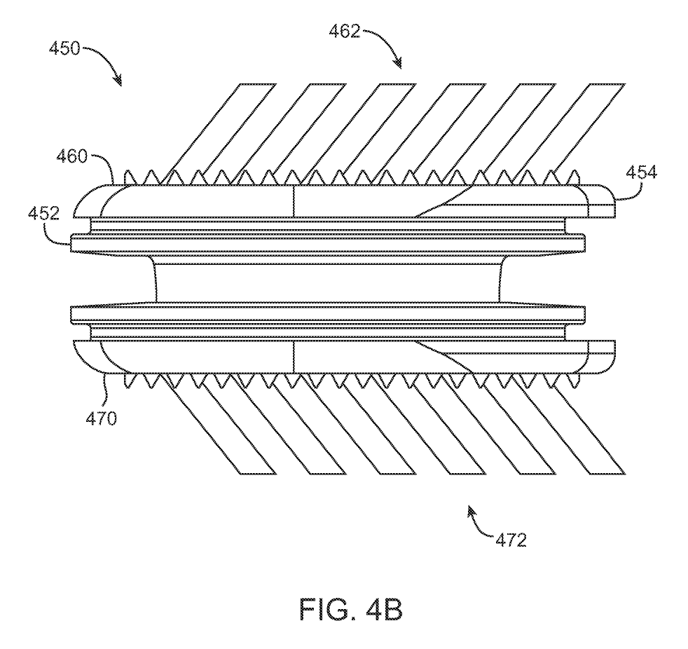

FIG. 4A schematically illustrates pillars inclined away from a midline on an intervertebral implant 400 according to an embodiment of the present invention. The pillars are arranged so that gaps are disposed between the pillars as described above. An upper row 414 of inclined pillars on an upper component 410 is inclined away from a midline 402 of the implant. A lower row 424 of inclined pillars disposed on a lower component 420 is inclined away from midline 402. Upper component 410 and lower component 420 are similar in other respects to the upper and lower components described above. For example upper component 410 includes serrations 112 and 113 and lower component 420 includes serrations 122 and 123.

FIG. 4B schematically illustrates pillars inclined proximally on an intervertebral implant 450 according to an embodiment of the present invention. The pillars are arranged so that gaps are disposed between the pillars as described above. Intervertebral implant 450 includes a distal end 452 that leads as the implant is inserted into the intervertebral space, and a proximal end 454 that follows the distal end as the implant is inserted into the intervertebral space. Intervertebral implant 450 includes an upper end plate 460 having a row of pillars 462 disposed thereon. Each pillar of row of pillars 462 is inclined proximally. Intervertebral implant 450 includes a lower endplate 470 having a row of pillars 472 disposed thereon. Each pillar of row of pillars 472 is inclined proximally. The components are similar in other respects to the components described above and are adapted to articulate.

Although illustrated figures and embodiments show specific configurations of rows of pillars, many other combinations are possible. For example, rows of pillars can be positioned in any location where an elongate anchor, fin or keel has been used in prior devices. Such devices include U.S. Pat. Nos. 5,314,477; 6,740,118; and 6,936,071; and U.S. Pat. Pub. Nos. 2005/0192586 and 2005/0197706, the full disclosures of which are incorporated herein by reference. Also, the pillars as described herein can be used with other intervertebral prostheses that restore motion with articulate endplates, for example as described in U.S. Pat. Nos. 4,759,766 and 4,997,432.

While the exemplary embodiments have been described in some detail for clarity of understanding and by way of example, a variety of additional modifications, adaptations, and changes may be clear to those of skill in the art. Hence, the scope of the present invention is limited solely by the appended claims.

What is claimed is:

1. An intervertebral prosthesis comprising:
an upper component adapted to engage an upper vertebra;
a lower component adapted to engage a lower vertebra;
a row of pillars disposed on at least one of the upper component or the lower component, the row of pillars adapted to enter a groove formed in the upper vertebra or the lower vertebra and
wherein the upper component and the lower component are adapted to engage each other or an intermediate member to form an articulate joint between the vertebrae.

2. The prosthesis of claim 1 wherein the row of pillars includes gaps between the pillars to permit bone growth between the pillars.

3. The prosthesis of claim 1 wherein the row of pillars comprises from about 5 to 20 pillars.

4. The prosthesis of claim 1 wherein each pillar of the row has a base attached to the first component or the second component, and each pillar extends from the base to a tip.

5. The prosthesis of claim 1 wherein each pillar has a height and several pillars have substantially the same height.

6. The prosthesis of claim 1 wherein each pillar has a maximum cross sectional width at a base of the pillar and a height of the pillar from the base to the tip is at least the maximum cross sectional width at the base.

7. The prosthesis of claim 1 wherein the row of pillars includes pillars of increasing height along the row to facilitate insertion into the groove.

8. The prosthesis of claim 1 wherein at least some of the pillars have a tapered cross sectional width which increases in a proximal direction.

9. The prosthesis of claim 8 wherein the at least some of the pillars have a vertical recess on a distal surface.

10. The prosthesis of claim 9 wherein at least some of the pillars have a similar cross-sectional geometry.

11. The prosthesis of claim 9 wherein at least some of the pillars extend vertically.

12. The prosthesis of claim 9 wherein at least some of the pillars are inclined.

13. An intervertebral prosthesis comprising:
an upper component having a row of upper pillars disposed thereon, the upper row of pillars arranged to enter a groove in an upper vertebra;
a lower component having a row of lower pillars disposed thereon, the lower row of pillars arranged to enter a groove in a lower vertebra; and
wherein the upper component and the lower component are adapted to engaged each other or an intermediate member to form an articulate joint.

14. The prosthesis of claim 13 wherein the rows of pillars include gaps to permit bone growth between the pillars.

15. The prosthesis of claim 13 wherein each row has from about 5 to 20 pillars.

16. The prosthesis of claim 13 wherein at least one row of pillars includes pillars of increasing height along the row to facilitate insertion into the groove.

17. The prosthesis of claim 13 wherein each pillar of the upper row has a base attached to the upper component, and each pillar extends from the base to a tip.

18. The prosthesis of claim 13 wherein at least one component includes two rows of pillars adapted to enter two parallel vertebral grooves.

19. The prosthesis of claim 13 wherein each of several pillars has a tapered cross sectional width which increases in a proximal direction.

20. The prosthesis of claim 13 wherein the rows of pillars are offset from each other to avoid vertebral splitting.

21. The prosthesis of claim 20 wherein the prosthesis comprises a midline, and each row is disposed on an opposite side of the midline to offset the upper row from the lower row.

22. The prosthesis of claim 20 wherein the prosthesis comprises a midline and wherein one of the components has two rows of pillars with each row disposed on opposite sides of the midline and the other component has a single row of pillars disposed on the midline so that the rows are offset.

23. A method for anchoring an intervertebral prosthesis within an intervertebral space between a pair of vertebral bodies, said method comprising:
cutting a groove in at least one of the vertebral bodies; and
introducing the prosthesis into the intervertebral space so that a plurality of pillars on the prosthesis enters the groove to anchor the prosthesis to the at least one vertebrae.

24. The method as in claim 23, wherein the prosthesis comprises components arranged to articulate.

25. The method as in claim 23 wherein the plurality of pillars that enters the groove comprises from about 5 to 20 pillars adapted to enter the groove.

26. The method as in claim 23 further comprising releasing the prosthesis to articulate while the pillars are introduced into the groove.

27. The method of claim 25 wherein cutting comprises cutting a groove in each of the vertebral bodies so that each vertebral body is adapted to receive pillars.

28. An improved intervertebral prosthesis of the type including a first component adapted to engage a first vertebra, a second component adapted to engage a second vertebra, and an anchor on at least one of the first and second components which is axially oriented over a length of the component and is adapted to enter a groove formed in at least one of the vertebrae, the improvement comprising a plurality of axially aligned, spaced-apart anchors distributed over the length on the at least one component, wherein the plurality of anchors provide an increased surface area to receive and engage new bone growth relative to a single anchor.

29. The prosthesis of claim 28 wherein the plurality of anchors are disposed over a distance, and the plurality of anchors have increased surface area relative to the single anchor along the distance.

30. The prosthesis of claim 28 wherein the anchors are disposed on the first component and the second component and the anchors are adapted to enter a groove formed in each of the first and second vertebrae.
